(19) United States
(12) Patent Application Publication
(10) Pub. No.: US 2009/0222350 A1
(43) Pub. Date: Sep. 3, 2009

(54) METHODS, KITS AND METHOD OF MARKETING FOR INCREASING HAIR DIAMETER AND DELIVERING ADDITIONAL BENEFITS

(76) Inventors: Toshiyuki Iwata, Kobe (JP); Neil Jordan, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/393,330

(22) Filed: Feb. 26, 2009

Related U.S. Application Data
(60) Provisional application No. 61/067,684, filed on Feb. 29, 2008.

(51) Int. Cl. G06Q 30/00; A61K 8/49; A61K 8/81; A61K 8/42; A61Q 5/00; A61Q 5/12; G06Q 50/00

(52) U.S. Cl. 705/14; 424/70.1; 424/70.16

(57) ABSTRACT

Disclosed is a method for increasing hair diameter, comprising the steps of: topically applying a first composition comprising to a region where hair diameter increase is desired; and topically applying a second composition to a region where an additional benefit is desired; wherein the first composition comprises a hair diameter increasing agent comprising a xanthine compound, a vitamin B3 compound, and a panthenol compound; and wherein the second composition comprises an additional benefit agent, such additional benefit being selected from the group consisting of hair volume-up, hair conditioning, hair strengthening, scalp sensation, hair color, scent, and mixtures thereof. The kits and methods of the present invention increase hair diameter, and also provide: motivation to consumers to use such compositions for a longer period of time so that consumers can obtain hair diameter increase benefit; at least one additional benefit, while not-compromising the penetration of the hair diameter increasing agent; and/or optimized benefits from both hair diameter increasing agents and additional benefit agents.

This application claims the benefit of U.S. Provisional Application No. 61/067,684, filed on Feb. 29, 2008.

### FIELD OF THE INVENTION

The present invention relates to methods, kits and method of marketing for increasing hair diameter and also delivering at least one additional benefit.

### BACKGROUND OF THE INVENTION

Many attributes contribute to the appearance of hair considered to be attractive. For instance, hair with a full and thick appearance is very desirable, whether it be on the scalp, beard, or moustache regions. In contrast, hair with a thin appearance is not as attractive, and can even lead to a perception that the thin-haired individual is older than their chronological age. Furthermore, thin hair can be more difficult to style, and typically cannot be styled into as many hairstyles, leaving the individual frustrated and with an unkempt appearance. Because of the foregoing problems associated with thin hair, many thin-haired individuals expend great effort and time on grooming, yet still do not attain their desired hairstyle and appearance. This can lead to frustration and/or lack of confidence in his or her appearance. These problems can be experienced by both female and male consumers.

Accordingly, there is a need to provide consumers with a way to increase the fullness and thickness of hair appearance, thus resulting in younger-looking, more attractive hair appearance.

There exists a variety of products which attempt to meet the above need by, for example, increasing hair diameter. However, it takes a long time to make hair fibers increase diameters thereof. It may be difficult for consumers, who prefer to see an immediate effect, to use such product for a longer period of time. Thus, there exists a need for hair care compositions which increase hair diameters, and also motivate consumers to use such compositions for a longer period of time so that consumers can obtain hair diameter increase benefit.

There also exists a need for hair care compositions which increase hair diameters, and also provide at least one additional benefit. However, it is still difficult to obtain such additional benefits, while not-compromising the penetration of the hair diameter increasing agent. It is still difficult to obtain optimized benefits from hair diameter increasing agents and additional benefit agents.

Based on the foregoing, there remains a need for hair care compositions which increase hair diameters, and which also provide: motivation to consumers to use such compositions for a longer period of time so that consumers can obtain hair diameter increase benefit; at least one additional benefit, while not-compromising the penetration of the hair diameter increasing agent; and/or optimized benefits from both hair diameter increasing agents and additional benefit agents.

None of the existing art provides all of the advantages and benefits of the present invention.

### SUMMARY OF THE INVENTION

The present invention is directed to a method for increasing hair diameter, comprising the steps of:

topically applying a first composition comprising to a region where hair diameter increase is desired; and
topically applying a second composition to a region where an additional benefit is desired;
wherein the first composition comprises a hair diameter increasing agent comprising:

a. a xanthine compound;

b. a vitamin B3 compound; and

c. a panthenol compound; and

wherein the second composition comprises an additional benefit agent, such additional benefit is selected from the group consisting of hair volume-up, hair conditioning, hair strengthening, scalp sensation, hair color, scent, and mixtures thereof.

The present invention is also directed to a kit for increasing hair diameter, comprising a first and a second compositions packaged in different containers, wherein the first composition comprises a hair diameter increasing agent comprising:

a. a xanthine compound;

b. a vitamin B3 compound; and

c. a panthenol compound; and

wherein the second composition comprises an additional benefit agent, such additional benefit is selected from the group consisting of hair volume-up, hair conditioning, hair strengthening, scalp sensation, hair color, scent, and mixtures thereof.

The present invention is further directed to a method of marketing compositions for increasing hair diameter, wherein said method comprises:

(a) offering for sale of a first composition;
(b) offering for sale of a second composition; and
(c) recommending a combination use of said first and second compositions for increasing hair diameter;
wherein the first composition comprises a hair diameter increasing agent comprising:

a. a xanthine compound;

b. a vitamin B3 compound; and

c. a panthenol compound; and

wherein the second composition comprises an additional benefit agent, such additional benefit is selected from the group consisting of hair volume-up, hair conditioning, hair strengthening, scalp sensation, hair color, scent, and mixtures thereof.

The kits and methods of the present invention provide hair diameter increasing benefit. The kits and methods of the present invention also provide: motivation to consumers to use such compositions for a longer period of time so that consumers can obtain hair diameter increase benefit; at least one additional benefit, while not-compromising the penetration of the hair diameter increasing agent; and/or, optimized benefits from both hair diameter increasing agents and additional benefit agents.

These and other features, aspects, and advantages of the present invention will become better understood from a reading of the following description, and appended claims.

### DETAILED DESCRIPTION OF THE INVENTION

While the specification concludes with the claims particularly pointing and distinctly claiming the invention, it is believed that the present invention will be better understood from the following description.

All percentages, parts and ratios are based upon the total weight of the hair care compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

Herein, “comprising” means that other steps and other ingredients which do not affect the end result can be added. This term encompasses the terms “consisting off” and “consisting essentially of”.

Herein, “mixtures” is meant to include a simple combination of materials and any compounds that may result from their combination.

Herein, “hair diameter increase” and “increasing hair diameter” mean increasing diameters of new hairs. New hairs herein means, any portions of hairs which are newly generated in hair follicles and appeared over the surface of skin/scalp as a result of hair growth, which include, for example, tips of newly growing hairs and roots of existing hairs.

All measurements are understood to be made at 25° C. and at ambient conditions, where “ambient conditions” means conditions under about one atmosphere of pressure and at about 50% relative humidity.

### Compositions

As used herein, the term “compositions” and “hair care compositions” are compositions that are applied to the hair and/or the skin underneath the hair, including compositions used to treat or care for the hair. Products contemplated by the phrases “compositions” and “hair care composition” include, but are not limited to liquids, creams, wipes, hair conditioners (rinse-off and leave-on), hair and/or scalp tonics, shampoos, hair colorants, mousses, propellant lotions, emulsions, shave gels, after-shave tonics and lotions, temporary beard hair dyes, and the like. In some embodiments, leave-on hair care compositions are preferred among the above hair care compositions, in view of providing effectively delivering the benefits of the present invention especially hair diameter increase benefit. Such leave-on hair care compositions include, for example, leave-on hair conditioning compositions, hair and/or scalp tonics, hair styling compositions. The hair care compositions, especially leave-on hair care compositions can have a pH of, for example, from about 3 to about 7.5, preferably from about 3.5 to about 7, more preferably from about 4 to about 7.

First compositions of the present invention comprise a hair diameter increasing agent. Second compositions of the present invention comprise an additional benefit agent. Optionally, the compositions can contain a dermatologically-acceptable carrier and/or any desired suitable optional ingredients.

### A. First Composition Comprising Hair Diameter Increasing Agent

In the present invention, it may be preferred that the first composition is in a non-emulsion form. It is believed that, the hair diameter increasing agent of the present invention provide improved performance to scalp, skin, and/or hair follicles, when it is contained in a non-emulsion composition rather than in an emulsion composition.

Preferably, the first compositions of the present invention are substantially free of substantially insoluble oily compounds. It is believed that lower levels of such substantially insoluble oily components provide at least one of the followings:

improved penetration of hair diameter increasing agents to scalp, skin and/or hair follicles;
improved solid particulate formation of the deposition agent, especially when the deposition agents are those being soluble in the composition and forming solid particulate on the hair; and
improved usage feel such as improved clean feel, especially when the composition is for leave-on use.

In the present invention, the compositions being “substantially free” of substantially insoluble oily compound means that the composition includes 1.0% or less, preferably 0.5% or less, more preferably 0.1% or less, still more preferably 0.05% or less, even more preferably 0% by weight of substantially insoluble oily compounds. By “substantially insoluble” ingredients, what is meant in the present invention is that: the ingredient is substantially insoluble in the compositions at the level used; and, when containing the ingredient at the level used, the composition has a transmittance of below about 25%, preferably below about 35%, more preferably below about 50%, still more preferably below about 60%, even more preferably below about 70%, and further more preferably below about 80% at 25° C. The transmittances are measured at 600 nm using UV-1601 which is a UV-visible spectrophotometer available from Shimadzu. Such “substantially insoluble” oily compounds are typically those selected from hydrocarbons, fatty compounds, and mixtures thereof. Such hydrocarbons include, for example, poly α-olefin oils, paraffins, waxes, and mixtures thereof. Such fatty compounds include, for example, fatty alcohols such as cetyl alcohol and stearyl alcohol, fatty acids such as stearic acid, fatty alcohol derivatives and fatty acid derivatives such as esters and ethers thereof, and mixtures thereof.

### A-1. Hair Diameter Increasing Agent

The first composition comprises a hair diameter increasing agent. A safe and effective amount of the hair diameter increasing agent can be included in the composition. The term “safe and effective amount,” as used herein, means an amount of a compound or composition sufficient to increase the diameter of the hair in the subject region of hair, when used for a result-effective period of time, but low enough to avoid serious side effects, i.e., to provide a reasonable benefit to risk ratio, within the scope of sound judgment of the skilled artisan. For example, the hair diameter increasing agent can be included in the first composition at a level by weight of the composition of, from about 0.1%, preferably from about 0.3%, more preferably from about 0.5%, and to about 20%, preferably to about 15%, more preferably to about 10%, in view of usage feel and hair diameter increasing effect.

Hair diameter increasing agents useful herein are those being substantially soluble in the first composition, in order to provide hair diameter increasing benefit by penetrating scalp, skin and/or hair follicles. By “substantially soluble” ingredients, what is meant in the present invention is that: the ingredient is substantially soluble in the composition at the level used; and, when containing the ingredient at the level used, the composition has a transmittance of about 25% or more, preferably about 35% or more, more preferably about 50% or more, still more preferably about 60% or more, even more preferably about 70% or more, and further more preferably about 80% or more, at 25° C.

The hair diameter increasing agent of the present invention comprises a xanthine compound, a vitamin B3 compound, and a panthenol compound. Preferably, the hair diameter increasing agent comprises caffeine, niacinamide, and panthenol. The use of the three compounds as a mixture, compared to the use of each compound, provide improved hair diameter increasing benefit.

### A-1-1. Xanthine Compound

As used herein, “xanthine compound” means one or more xanthines, derivatives thereof, and mixtures thereof. Xanthine Compounds that can be useful herein include, but are not limited to, caffeine, xanthine, 1-methyl xanthine, theophylline, theobromine, derivatives thereof, and mixtures thereof. Among these compounds, caffeine is preferred in view of its solubility in the composition and hair diameter increasing effect. The composition can contain from about 0.1%, preferably from about 0.2%, more preferably from about 0.3%, and to about 10%, preferably to about 5%, more preferably to about 2% by weight of a xanthine compound, in view of usage feel and hair diameter increasing effect.

### A-1-2. Vitamin B3 Compound

As used herein, “vitamin B3 compound” means a one or more compounds having the formula:

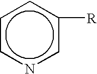

wherein R is —CONH₂ (i.e., niacinamide), —COOH (i.e., nicotinic acid) or —CH₂OH (i.e., nicotinyl alcohol); derivatives thereof; mixtures thereof; and salts of any of the foregoing.

Exemplary derivatives of the foregoing vitamin B3 compounds include nicotinic acid esters, including non-vasodilating esters of nicotinic acid (e.g, tocopherol nicotinate, and myristyl nicotinate), nicotinyl amino acids, nicotinyl alcohol esters of carboxylic acids, nicotinic acid N-oxide and niacinamide N-oxide.

One or more vitamin B3 compounds may be used herein. Preferred vitamin B3 compounds are niacinamide and tocopherol nicotinate. Niacinamide is more preferred, in view of usage feel, its solubility in the composition, and hair diameter increasing effect. It is believed that, niacinamide can stay on scalp, pore and hair, where cells contribute to hair diameter increase exist, for a longer period of time and thus can provide pro-longed benefit, while other vitamine B3 products such as nicotinates may penetrate fast into scalp, and thus may move away from such cells. It is also believed that, niacinamide can be stable in the composition and when applied for a longer period of time, while other vitamine B3 products such as nicotinic acid esters may be decomposed to nicotinic acids, and thus may cause skin flushing and itchiness. Such nicotinic acid esters include, for example, ethyl nicotinate, octyl nicotinate, myristyl nicotinate, and cetyl nicotinate. When used, salts, derivatives, and salt derivatives of niacinamide are preferably those having substantially the same efficacy as niacinamide.

In a preferred embodiment, the ring nitrogen of the vitamin B3 compound is substantially chemically free (e.g., unbound and/or unhindered), or after delivery to the skin becomes substantially chemically free (“chemically free” is hereinafter alternatively referred to as “uncomplexed”). More preferably, the vitamin B3 compound is essentially uncomplexed. Therefore, if the composition contains the vitamin B3 compound in a salt or otherwise complexed form, such complex is preferably substantially reversible, more preferably essentially reversible, upon delivery of the composition to the skin. For example, such complex should be substantially reversible at a pH of from about 5.0 to about 6.0. Such reversibility can be readily determined by one having ordinary skill in the art.

More preferably the vitamin B3 compound is substantially uncomplexed in the composition prior to delivery to the keratinous tissue. Exemplary approaches to minimizing or preventing the formation of undesirable complexes include omission of materials which form substantially irreversible or other complexes with the vitamin B3 compound, pH adjustment, ionic strength adjustment, the use of surfactants, and formulating wherein the vitamin B3 compound and materials which complex therewith are in different phases. Such approaches are well within the level of ordinary skill in the art.

Thus, in a preferred embodiment, the vitamin B3 compound contains a limited amount of the salt form and is more preferably substantially free of salts of a vitamin B3 compound. Preferably the vitamin B3 compound contains less than about 50% of such salt, and is more preferably essentially free of the salt form. The vitamin B3 compound in the compositions hereof having a pH of from about 4 to about 7 typically contain less than about 50% of the salt form.

The composition can contain from about 0.1%, preferably from about 0.5%, more preferably from about 1%, and to about 25%, preferably to about 15%, more preferably to about 7.5% by weight of a vitamin B3 compound, in view of usage feel and hair diameter increasing effect.

### A-1-3. Panthenol Compound

As used herein, the term “panthenol compound” is broad enough to include panthenol, one or more pantothenic acid derivatives, and mixtures thereof. Panthenol and its derivatives can include D-panthenol ([R]-2,4-dihydroxy-N-[3-hydroxypropyl)]-3,3-dimethylbutamide), DL-pan-thenol, pantothenic acids and their salts, preferably the calcium salt, panthenyl triacetate, royal jelly, panthetine, pantotheine, panthenyl ethyl ether, pangamic acid, pantoyl lactose, Vitamin B complex, or mixtures thereof.

Compositions comprising pantothenic acid derivatives that remain more stable than panthenol and other similar materials in acidic compositions or in compositions containing acid-producing materials such as aluminum-containing actives, can also be suitable for use herein. The selected pantothenic acid derivatives are most typically in liquid form and dispersed throughout or otherwise solubilized within the liquid carrier component of the composition.

The term “pantothenic acid derivative” as used herein refers to those materials that conform to the formula:

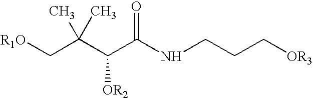

wherein R₁, R₂ and R₃ are hydrogen, C2-C20 hydrocarbons, C2-C20 carboxylic acid esters, or combinations thereof, provided that not more than two of R₁, R₂ and R₃ are hydrogen. In one embodiment, R₁, R₂ and R₃ are independently selected from hydrogen, C2-C8 hydrocarbons, C2-C8 carboxylic acid esters, or combinations thereof, in another embodiment, R₁ and R₂ are hydrogen, and R₃ is a C2-C8 hydrocarbon, C2-C8 carboxylic acid ester, or combinations thereof, in yet another embodiment, R₁ and R₂ are hydrogen and R₃ is ethyl. The selected pantothenic acid derivatives may be derived or otherwise obtained from any known source, which may include pantothenic acid or materials other than pantothenic acid, so long as the resulting material has the above defined chemical formula.

Specific non-limiting examples of selected pantothenic acid derivatives for use herein include ethyl panthenol, panthenyl triacetate, and combinations thereof. In a particular embodiment, a pantothenic acid derivative comprises the d-isomeric form(s) of such derivative form(s), such as d-ethyl panthenol.

Among these compounds, D-panthenol is preferred in view of its solubility in the composition and hair diameter increasing effect.

The composition can contain from about 0.01%, preferably from about 0.02%, more preferably from about 0.05%, and to about 3%, preferably to about 1%, more preferably to about 0.5% by weight of a panthenol compound, in view of usage feel and hair diameter increasing effect.

### A-2. Thickener

Preferably, the first compositions further comprise a thickener such that the composition does not drip undesirably onto other areas of the body, onto clothing, or onto home furnishings. Such compositions can have a zero shear viscosity of from about 0.1 Pa·s, preferably from about 10 Pa·s, more preferably from about 100 Pa·s, and to about 100,000 Pa·s, preferably to about 50,000 Pa·s, more preferably to about 10,000 Pa·s. Generally, the viscosity of the personal care composition decreases according to the increase of shear stress. At a lower shear stress, the viscosity of the composition does not change and this is called “First Newtonian Plateau”. In the shear stress range higher than a yield point, the viscosity of the composition decreases. The zero shear viscosity is the average of the viscosity measured in the first Newtonian plateau. The zero shear viscosity is measured at 25° C. by shear stress ramp mode using AR2000 available from TA Instruments.

Any suitable thickener can be used, for example: a cellulose-based thickener such as methyl cellulose, ethyl cellulose, hydroxyethyl cellulose, and hydroxypropylmethyl cellulose; a crosslinked acrylic acid homopolymer with the CTFA name Carbomer; and a non-cellulose based amphiphilic polymer which comprises at least one fatty chain and non-cellulose based hydrophilic moieties, and which is preferably crosslinked.

Non-cellulose based amphiphilic polymers are preferably used. Such non-cellulose based amphiphilic polymers include, for example, carboxylic acid/carboxylate copolymers which are obtained by copolymerizing: 1) a carboxylic acid monomer such as acrylic acid, methacrylic acid, maleic acid, maleic anhydride, itaconic acid, fumaric acid, crotonic acid, or α-chloroacrylic acid, preferably acrylic acid; 2) a carboxylic ester having an alkyl chain of from 1 to about 30 carbons, preferably acrylic esters having an alkyl chain of from 10 to about 30 carbons; and preferably 3) a crosslinking agent such as allyl ethers of sucrose and allyl ethers of pentaerythritol. Highly preferred examples of such non-cellulose based amphiphilic polymers include Acrylates/C10-30 Alkyl Acrylate Crosspolymer.

Preferred are those being substantially soluble in the composition, in view of not-compromising the penetration of hair diameter increasing agent. In the present invention, Acrylates/C10-30 Alkyl Acrylate Crosspolymer is preferred especially when the composition comprises higher level of alcohols such as ethanol, in view of better solubility in such compositions compared to other anionic/nonionic thickeners such as carbomer and cellulose polymers. Acrylates/C10-30 Alkyl Acrylate Crosspolymer is also preferred in view of hair feel when dried, compared to cationic/nonionic thickening polymers. Acrylates/C10-30 Alkyl Acrylate Crosspolymer can be used together with any neutralizer such as triethanolamine.

Such thickeners can be included in the composition, at a level by weight of the composition of from about 0.01%, preferably from about 0.05%, and to about 5%, preferably to about 3%, more preferably to about 1%, in view of thickening effect, feel during and/or after usage, and/or solubility in the composition.

### A-3. Substantially Soluble Silicone Copolyol Conditioning Agent

The first composition may further contain a substantially soluble silicone conditioning agent. Such silicone conditioning agents can be included in the composition, at a level by weight of the composition of from about 0.05%, preferably from about 0.1%, more preferably from about 0.2%, and to about 10%, preferably to about 5%, more preferably to about 2%, in view of conditioning benefit and clean dry feel.

Such silicone conditioning agents useful herein are composition, for example: silicone copolyol conditioning agents; and anionic silicone conditioning agents such as Acrylates/Behenyl Acrylate/Dimethicone Methacrylate Copolymer, Actylic Acid/Stearyl Methacrylate/Dimethicone Methacrylate Copolymer. Preferably, silicone conditioning agents useful herein are silicone copolyol conditioning agents. It is believed that, compared to other soluble conditioning compounds such as fatty esters and fatty ethers, substantially silicone conditioning agents especially substantially soluble silicone copolyol conditioning agents can provide conditioning benefits while providing better clean dry feel and not-compromising the penetration of hair diameter increasing agent.

Among such silicone copolyol conditioning agents, preferred are nonionic silicone copolyol conditioning agents, especially when thickener is an anionic polymer such as Acrylates/C10-30 Alkyl Acrylate Crosspolymer, in view of compatibility with such thickeners. Such nonionic silicone copolyol conditioning agents are those being substantially free of cationic, amido and amino substitutions. What meant by being “substantially free of cationic, amido and amino substitutions” is that the silicone compounds contains 1% or less, preferably 0.5% or less, more preferably 0% by weight of such substitutions.

Such silicone copolyols useful herein include, for example, dimethicone copolyols dimethicone backbone and polyoxyalkylene substitutions such as polyethylene oxide or/and polypropylene oxide substitutions which are grafted to the middle and/or the ends of the dimethicone backbone. In view of the solubility in the compositions, graft silicone copolyols are preferred compared to silicone copolyols those having the substitutions only at the ends of the backbone. The grafted silicone copolyols useful herein have an HLB value of preferably from about 5 to about 17, more preferably from about 8 to about 17, still more preferably from about 8 to about 12. The grafted silicone copolyols having the same INCI name have a variety of the weight ratio, depending on the molecular weight of the silicone portion and the number of the polyethylene oxide or/and polypropylene oxide substitutions.

Commercially available grafted dimethicone copolyols include, for example: those having a tradename Silsoft 430 having an HLB value of from about 9 to about 12 (INCI name “PEG/PPG-20/23 dimethicone”) available from Momentive Performance Materials; those having a tradename Silsoft 475 having an HLB value of from about 13 to about 17 (INCI name “PEG-23/PPG-6 dimethicone”); those having a tradename Silsoft 880 having an HLB value of from about 13 to about 17 (INCI name “PEG-12 dimethicone”); those having a tradename Silsoft 440 having an HLB value of from about 9 to about 12 (INCI name “PEG-20/PPG-23 dimethicone”); those having a tradename DC5330 (INCI name “PEG-15/PPG-15 dimethicone”) available from Dow Corning. Among them, preferred are those having a tradename Silsoft 430 having an HLB value of from about 9 to about 12 (INCI name “PEG/PPG-20/23 dimethicone”) available from Momentive Performance Materials.

### B. Second Composition Comprising Additional Benefit Agent

The second compositions of the present invention comprise an additional benefit agent. Such additional benefit agent is selected from the group consisting of hair volume-up agent, hair conditioning agent, hair strengthening agent, scalp sensation agent, hair color agent, scent agent, and mixtures thereof. It is believed that, by the use of such additional benefit agent, consumers are motivated to use the composition containing hair diameter increasing agent for a longer period of time, thus, can obtain the benefit from the hair diameter increasing agent.

Such additional benefit agent can be incorporated into the composition at a level by weight of the composition of from about 0.01%, preferably from about 0.05%, more preferably from about 0.1%, and to about 20%, preferably to about 15%, more preferably to about 10%.

Hair volume-up agents useful herein include, for example, agents which provide at least one of the following: hair hold, hair root lift, scalp coverage, hair coverage, and mixtures thereof. Such agents useful herein include, for example: film forming polymers which can hold hairs and/or lift hair root, such polymers including, for example, PVP, VP/VA Copolymer, Acrylates/VA Copolymer, Acrylates/Acrylamide Copolymer, Chitosan, Polyacrylate-6, Polyacrylate-15, Polyquatemium-68, VP/Crotonates/Vinyl Propionate Copolymer, VP/Methacrylamide/Vinyl Imidazole Copolymer; solid particulates which can lift hair root by depositing on hair root, such particulates including, for example, silica, mica, bentonite, clay, polyethylene spherical particle, PMMA spherical particle, Polymethylsilsesquioxane spherical particle, glass, silicone wax, mineral wax, colored solid particulates or fibers which can cover scalp or hair, such particulates or fibers including, for example, mica, pigment, iron oxide, cellulose, rayon, nylon, wool, silk, all having a color similar to color of the hair. Above solid particulates and fibers can be used together with adhesive materials.

Hair conditioning agents useful herein include, for example, agents which provide at least one of the following: hair shine, hair luster, hair softness, hair smoothness, hair moisturization, hair friction reduction, ease to comb hair, and mixtures thereof. Such agents useful herein include, for example: cationic surfactants including, for example, mono- or di-alkyl quaternized ammonium salts such as behenyl trimethyl ammonium methosulfate and distearyl dimethyl ammonium chloride, and amidoamines such behenamidopropyldimethylamine and salts thereof; cationic polymers such as cationic celluloses, cationic guars, and other synthesized cationic polymers; fatty compounds including, for example, fatty alcohols such as cetyl alcohol, stearyl alcohol and behenyl alcohol; silicones such as dimethicone, dimethiconol, dimethicone copolyol, aminosilicones, and aminosilicone copolyols.

Hair strengthening agents useful herein include, for example, agents which provide at least one of the following: hair breakage reduction, hair tensile strength increase, hair flexural modulus increase, and mixtures thereof. Such agents useful herein include, for example: a combination of (a) an aminosilicone having a viscosity of from about 1,000 centistokes to about 1,000,000 centistokes, and less than about 0.5% nitrogen by weight of the aminosilicone, (b) a silicone copolymer emulsion with an internal phase viscosity of greater than about 120×106 centistokes, as measured at 25° C., and (c) optionally, a polyalkylsiloxane mixture comprising a first polyalkylsiloxane which is non-volatile, substantially free of amino groups, and has a viscosity of from about 100,000 mm²·s⁻¹ to about 30,000,000 mm²·s⁻¹ and a second polyalkylsiloxane which is non-volatile, substantially free of amino groups, and has a viscosity of from about 5 mm²·s⁻¹ to about 10,000 mm²·s⁻¹; and a combination of (a) a silicone polymer containing quaternary groups wherein said silicone polymer comprises silicone blocks with greater than about 200 siloxane units; (b) a silicone copolymer emulsion with an internal phase viscosity of greater than about 120×106 centistokes, as measured at 25° C., and (c) optionally, a silicone copolyol. While the above combinations of silicones are preferably used, other agents such as hydroxyethyl urea and glycerin can be used for increasing tensile strength.

Scalp sensation agents useful herein include, for example, agents which provide at least one of the following: scalp cooling, scalp warming, and mixtures thereof. Such agents useful herein include, for example: menthol, menthoxypropanediol, capsaicin, and vanillyl butyl ether.

Among the above additional benefit agents, preferred are as follows:

hair volume-up agents, in view of providing immediate hair volume-up benefits which may be expected by consumers who use hair diameter increasing agents/products;
hair conditioning agents, in view of providing hair conditioning benefit which may be expected by consumers who use hair care compositions; and
hair strengthening agents, in view of providing hair strengthening benefit which may be expected by consumers who use hair diameter increasing agents/products.

### C. Carrie

Preferably, the first/second compositions of the present invention further comprise a carrier. Carriers useful herein are dermatologically-acceptable carriers. The term “dermatologically-acceptable,” as used herein, means that the compositions or components thereof so described are suitable for use in contact with mammalian keratinous tissue without undue toxicity, incompatibility, instability, allergic response, and the like. Such dermatologically-acceptable carriers useful herein include, for example, aqueous carriers such water and water solutions of lower alkyl alcohols and polyhydric alcohols. The lower alkyl alcohols useful herein are monohydric alcohols having 1 to 6 carbons, preferably ethanol and isopropanol, more preferably ethanol. The polyhydric alcohols useful herein include propylene glycol, hexylene glycol, glycerin, and propane diol. Generally, the compositions of the present invention comprise from about 10%, preferably from about 30%, more preferably from about 80%, and to about 99%, preferably to about 95%, more preferably to about 95% by weight of such aqueous carriers.

Preferably, the first composition comprises an aqueous carrier comprising water and alcohols such as ethanol. Such alcohols such as ethanol can be included at a level by weight of the composition, of from 10%, preferably from 20%, more preferably from 30%, still more preferably from 40%, and to 90%, preferably to 70%, more preferably to 60%, in view of providing clean usage feel, improved solubilization and penetration of hair diameter increasing agents especially caffeine, niacinamide and panthenol.

### D. Optional Ingredients

The first and second compositions of the present invention may include other optional ingredients, which may be selected by the artisan according to the desired characteristics of the final product and which are suitable for rendering the composition more cosmetically or aesthetically acceptable or to provide them with additional usage benefits. Such optional ingredients can be incorporated into the composition at a level of from about 0.01% to about 10%, preferably from about 0.05% to about 5% by weight of the composition.

A wide variety of other additional components can be formulated into the present compositions. These include: hair growth agents such as minoxidil; anti-dandruff agents such as zinc pyrithione; conditioning agents such as hydrolysed collagen, vitamins, hydrolysed keratins, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; and ultraviolet and infrared screening and absorbing agents such as octyl salicylate.

### Method of Use/Method for Increasing Hair Diameter

The present invention also provides a method for increasing the diameter of the hair. Such methods comprise the steps of:

topically applying a safe and effective amount of the first composition comprising to a region where hair diameter increase is desired; and
topically applying a safe and effective amount of the second composition to a region where an additional benefit is desired.
The first and second compositions are explained above in detail.

The region where hair diameter increase benefit from the first composition is desired includes, for example, scalp and/or face (e.g., beard or moustache area). The region of hair can be located on any part of the body. For instance, it can grow from a skin surface located on at least a portion of the scalp or the face.

The region where additional benefits from the second composition is desired includes, for example, same region in which the first composition is applied, broader region including the region in which the first composition is applied, and different region from that in which the first composition is applied.

In a preferred embodiment, the first composition is applied to mainly scalp and the second composition is applied to mainly hair.

It is preferred that an effective amount of a composition of the present invention is topically applied to the desired region at effective frequency over a result-effective period of time.

The term “effective amount,” as used herein, means an amount of a compound or composition sufficient to increase the diameter of the hair in the subject region of hair. It depends on the product form and concentration of ingredients. For example, the first and second composition can be applied at an amount of, respectively, from about 0.1 ml to about 10 ml to the subject regions.

The term “effective frequency” as used herein means a frequency to apply the composition sufficient to increase the diameter of the hair in the subject region of hair. It depends on the product form and concentration of ingredients. For example, the first and second composition can be applied, respectively, at least once a few week, more preferably at least once a week, more preferably at least once a day, and up to about three times a day.

The term “result-effective period of time,” as used herein, means a period of time sufficient to increase the diameter of the hair in the subject region of hair. It depends on the product form and concentration of the hair diameter increasing agent. For example, the first and second compositions can be applied, respectively, for at least a week, more preferably at least two weeks, still more preferably at least four weeks.

In still another embodiment, the method comprises applying the composition according to a regimen, wherein said regimen comprises:

(a) cleansing the scalp and/or face to form a cleansed scalp and/or face;

(b) topically applying the first composition to said cleansed scalp and/or cleansed face;

(c) topically applying the second composition to a region where an additional benefit is desired.

### Kit

The present invention further provides a kit for increasing the diameter of the hair. Such kits comprise the first and the second compositions packaged in different containers. The first and second compositions are explained above in detail.

Such containers can be made by plastic and/or glass usually. The containers useful herein include, for example, a bottle having a closure on the top of the bottle, an upside down bottle having a closure at the bottom of the bottle, a bottle with a pump dispenser, a bottle with a foaming dispenser, and a tube with a closure.

The kit can also be contained in an outer container in which the first composition container and the second composition container are packaged together. Such outer container can be made by plastic or paper.

The kits of the present invention can further contain recommendation of a combination use of said first and second compositions for increasing hair diameter. Such recommendations can be contained by, for example, describing on at least one of the containers.

### Method of Marketing

The present invention also relates to a method of marketing compositions for increasing the diameter of the hair. Such methods of marketing comprise:

(a) offering for sale of the first composition;
(b) offering for sale of the second composition; and
(c) recommending a combination use of said first and second compositions for increasing hair diameter;

The first and second compositions are explained above in detail. The recommendation can be, for example, TV advertisement, video advertisement in stores, written recommendations near and/or on store shelves where the compositions are displayed, and or written recommendations on containers in which the compositions are packaged.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Example 1

### Clinical Study

A composition comprising a mixture of Caffeine, Niacinamide and Panthenol demonstrated a clinically significant increase in hair width versus a placebo. In this study, a scalp tonic containing 5% Niacinamide, 1.5% Caffeine, and 0.3% D-Panthenol in a water/alcohol matrix (25% ethanol, q.s. water) was evaluated. This formula also included 0.3% hydroxypropylmethylcellulose as a thickening agent. In this split-head study, 1.5 ml of the tonic was applied to one side of the head two times per day, and 1.5 ml of the placebo was applied to the other side of the head two times daily.

This was a 14 week, double blinded, randomized, split-head and controlled clinical study in women ages 18-65 (inclusive) who perceived themselves as having thinning hair. This study consisted of a 2 week pre-treatment phase (use of placebo on both sides of the head to learn proper product application methods prior to the actual treatment phase) followed by a 12 week treatment phase.

Upon arriving at the test site, subjects were acclimated in an environmentally controlled room (70° F.±2 and 30-45% relative humidity) for 30 minutes prior to having their scalp evaluated by a qualified skin grader to assess skin health. After subjects had their scalp assessed by a qualified skin grader, a licensed cosmetologist identified two scalp sites for clipping (areas of approximately 1.8 cm×1.8 cm), one on the top left lateral scalp, approximately 4-5″ back into the hairline and the other was symmetrically placed on the top right lateral scalp. A template was used to mark the 4 corners and a notch slightly below center of each treatment site. The clipped sites were approximately 2″ lateral to either of their respective sides from the midline of the head/scalp. The licensed cosmetologist then cut the hair within each of the scalp clipping sites with a small battery operated hair clipper to about 1 mm in length. After the sites were prepared, a trained clinical assistant placed a small, permanent dot tattoo approximately 1 mm above the marked notch (located slightly below center of the treatment site) on each treatment site to provide a reference for identification of these sites for digital imaging that was performed at designated study time points.

For Baseline, Week 4, 8, and 12, the clipped scalp sites were identified using the template and marking the 4 corners of the template with a Sharpie® marker or equivalent. The licensed cosmetologist then evenly clipped the short hairs within the 2 clipped sites to 1 mm as previously conducted. Following hair clipping, a technician captured a baseline image of each clipped scalp site using a Hi-Scope® digitized imaging system. The tattoo that was placed within each lower treatment site was used as a reference point to position the image for capture and storage. This baseline image was used when blending post-treatment images during the study. The next day, approximately 24 hours following the hair clipping visit, a technician captured an image of each lower scalp clipping site using a Hi-Scope® digitized imaging system. The tattoo that was placed within each lower treatment site was used as a reference point to position the image for capture and storage after aligning the hair follicle openings with the image captured at Baseline, Day 1. This procedure was used for Week 4, 8 and 12.

A significant increase in hair diameter was observed for the hair tonic treated hair vs. the placebo-treated hair in the human clinical test. The Change from Baseline Adjusted Mean for the tonic-treated hair (0.663 μm) is larger than that of the placebo-treated (0.165 μm). As this is a split-head design, subject variation was greatly minimized. Additionally, since the exact same hairs were imaged throughout the study, variations in width due to measuring different hairs through the study were greatly minimized.

### Example 1

### Clinical Study—Comparison of Treatment Effect

| | | | Change from Baseline |
|---|---|---|---|
| N | Treatment | Grouping | Adjusted Mean (μm) |
| 40 | Placebo | A | 0.165 |
| | (Tonic Vehicle) | | |
| 40 | Tonic Product | B | 0.663 |

An image analysis algorithm was used to measure the hair diameter from the digital micrographs. The algorithm automatically carried out several steps to isolate and characterize hairs in each digital image. First, the algorithm performed a local background leveling function and enhancement of the red component in the color image. Automatic thresholding of the color image was performed to obtain a binary image (black and white) of the hairs on the background. The algorithm then identified hairs as continuous black lines and broke the lines into individual hairs at branch points and points of high curvature. Next, the algorithm eliminated very short objects and retained the remaining objects as ‘hairs’. In the final step, the algorithm measured and recorded position and diameter (width) of each detected hair.

The hair system image acquisition and analysis methods used herein were the methods published by Berger, R. S. et al., in 2003 in “The effects of minoxidil, 1% pyrithione zinc and a combination of both on hair density: a randomized controlled trial. British Journal of Dermatology, 149, 354-362,” with the exception that for the study of this example, different treatments, scalp imaging sites, and timepoints were used.

### Example 2

### Synergy Study

A mixture of Caffeine, Niacinamide and Panthenol demonstrated a surprising increase in the survival of metabolically stressed dermal papilla cells, versus individual components and versus mixtures of two of them, as shown below. The dermal papilla, in normal human hair, is the control center for hair diameter. Increasing survival of dermal papilla cells in situ leads to increased hair diameter. Accordingly, an increase in survival of dermal papilla cells correlates with an increase in hair diameter.

Primary human dermal papilla cells were metabolically stressed for 48 hours in reduced standard tissue culture media. During this period of stress the cells were treated with the mixtures or individual ingredients below. After 48 hr the metabolic activity of the cells was measured by the amount of ATP using the Cell Titer Glo™ kit (Promega).

| | Survival rate: |
|---|---|
| | % Change from |
| Treatment | Control |
| Control (100% water) | — |
| Niacinamide (0.006%) | −8% |
| Panthenol (0.15%) | +6% |
| Caffeine (0.05%) | +41% |
| Mixture of Niacinamide (0.006%) and Panthenol (0.15%) | +22% |
| Mixture of Niacinamide (0.006%) and Caffeine (0.05%) | +82% |
| Mixture of Panthenol (0.15%) and Caffeine (0.05%) | +93% |
| Mixture of Niacinamide (0.006%), Panthenol (0.15%), | +155% |
| and Caffeine (0.05%) | |

### Example 3

### Hair Care Composition Examples

First composition - Tonics

| Component (Active weight %) | A | B | C |
|---|---|---|---|
| Caffeine | 1.5 | 0.6 | — |
| Caffeine Carboxylic Acid | — | — | 1.00 |
| Niacinamide | 5.0 | 2.0 | — |
| Tocopherol Nicotinate | — | — | 1.0 |
| Panthenol | 0.3 | 0.1 | — |
| Panthenyl Ethyl Ether | — | — | 0.2 |
| PEG/PPG-20/23 Dimethicone | — | 0.7 | 0.5 |
| Carbomer | 0.1 | — | — |
| Acrylates/C10-30 Alkyl | — | 0.3 | 0.3 |
| Acrylate Crosspolymer | | | |
| Triethanolamine | 0.2 | 0.04 | 0.04 |
| Ethanol | 25.0 | 50.0 | 50.0 |
| Perfume | 0.1 | 0.1 | 0.5 |
| Tocopherol | — | — | 0.1 |
| Ascorbic Acid | — | — | 0.05 |
| Pentapeptide-2 | — | — | 0.05 |
| Deionized water | Qs | Qs | Qs |

Second composition - Mousse for styling/volume-up

| Component (Active weight %) | D | E | F |
|---|---|---|---|
| Glycerin | 5.0 | 1.0 | 1.0 |
| Carbomer | — | 0.1 | 0.1 |
| Triethanolamine | — | 0.2 | 0.2 |
| Polyacrylamide | 1.0 | — | — |
| Polyquaternium-68 | 1.5 | — | — |
| PVP | — | 0.2 | 0.2 |
| Ethylhexyl Hexadecanoate | 0.2 | 0.3 | 0.2 |
| 1,3-Butylene Glycol | 2.0 | 5.0 | 5.0 |
| Polysorbate 20 | 1.0 | 1.5 | 1.5 |
| Propellant | 6.0 | 6.0 | 6.0 |
| Ethanol | 3.0 | 1.0 | — |
| EDTA | 0.1 | 0.1 | 0.1 |
| Preservatives | 0.2 | 0.6 | 1.2 |
| Deionized water | Qs | Qs | Qs |

Second composition - Conditioner

| Component (Active weight %) | G |
|---|---|
| Stearamidopropyldimethylamine | 0.5 |
| Ditallow dimethyl ammonium chloride | 0.5 |
| Dimethicone gum/volatile silicone blend | 4.0 |
| Cetyl alcohol | 1.0 |
| Stearyl alcohol | 0.5 |
| Oleyl alcohol | 0.2 |
| Polysorbate-60 | 0.1 |
| Citric acid | 0.1 |
| Preservatives | 0.4 |
| Deionized water | Qs |

### Method of Preparation

The compositions of Examples A through G as shown above can be prepared by any conventional method well known in the art. For example, the compositions of Examples A through F are suitably made as follows:

The polymeric materials, if present, are dispersed in water at room temperature, mixed by vigorous agitation. Ethanol, if included, is added to the mixture. The hair diameter increasing agents, deposition agents, adhesive agents are added to the mixture with agitation to dissolve or disperse such components. Then the remaining components such as silicones and perfumes, if included, are added to the mixture with agitation. When the compositions are mousse, the mixture is packed into aerosol cans with propellants so that the final compositions for Examples D through F are obtained.

For example, the composition of Example G is suitable made as follows:

Polymeric materials, if included, are added to water with agitation, and heated to about 80° C. Cationic surfactants and high melting point fatty compounds are added to the mixture at about 80° C. with agitation. The mixture is cooled down to about 55° C. Silicone and, if included, additional components such as perfumes and preservatives are added to the mixture with agitation. Then the mixture is cooled down to room temperature.

Examples A through G are hair care compositions of the present invention which are particularly useful for leave-on use. The first compositions of Examples A through C are combined with any of the second compositions of Examples D through F, for the methods and kits of the present invention. The first compositions are topically applied to a region where hair diameter increase is desired. The second compositions are topically applied a region where an additional benefit is desired. These examples have many advantages. For example, the kits and methods of the present invention increase hair diameter, and also provide: motivation to consumers to use such compositions for a longer period of time so that consumers can obtain hair diameter increase benefit; at least one additional benefit, while not-compromising the penetration of the hair diameter increasing agent; and or optimized benefits from both hair diameter increasing agents and additional benefit agents. Such additional benefits are, for example, hair volume-up benefit when the second composition is any of Examples D through F, and hair conditioning benefit when the second composition is Example F.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for increasing hair diameter, comprising the steps of:
topically applying a first composition comprising to a region where hair diameter increase is desired; and
topically applying a second composition to a region where an additional benefit is desired;
wherein the first composition comprises a hair diameter increasing agent comprising:
a. a xanthine compound;
b. a vitamin B3 compound; and
c. a panthenol compound; and
wherein the second composition comprises an additional benefit agent, such additional benefit is selected from the group consisting of hair volume-up, hair conditioning, hair strengthening, scalp sensation, hair color, scent, and mixtures thereof.

2. A kit for increasing hair diameter, comprising a first and a second compositions packaged in different containers, wherein the first composition comprises a hair diameter increasing agent comprising:
a. a xanthine compound;
b. a vitamin B3 compound; and
c. a panthenol compound; and
wherein the second composition comprises an additional benefit agent, such additional benefit is selected from the group consisting of hair volume-up, hair conditioning, hair strengthening, scalp sensation, hair color, scent, and mixtures thereof.

3. A method of marketing compositions for increasing hair diameter, wherein said method comprises:
(a) offering for sale of a first composition;
(b) offering for sale of a second composition; and
(c) recommending a combination use of said first and second compositions for increasing hair diameter;
wherein the first composition comprises a hair diameter increasing agent comprising:
a. a xanthine compound;
b. a vitamin B3 compound; and
c. a panthenol compound; and
wherein the second composition comprises an additional benefit agent, such additional benefit is selected from the group consisting of hair volume-up, hair conditioning, hair strengthening, scalp sensation, hair color, scent, and mixtures thereof.

4. The composition of any of the claims 1-3, wherein additional benefit is selected from the group consisting of hair volume-up, hair conditioning, hair strengthening, and mixtures thereof.

5. The composition of any of the claims 1-3, wherein said xanthine compound comprises caffeine, said vitamin B3 compound comprises niacinamide, and said panthenol compound comprises panthenol.

6. The composition of claim 5, wherein the xanthine compound is present in the composition at a level of from about 0.1% to about 10%, the vitamin B3 compound is present in the composition at a level of from about 0.1% to about 25%, and the panthenol compound is present in the composition at a level of from about 0.01% to about 3%

7. The composition of claim 6, wherein the xanthine compound is caffeine, the vitamin B3 compound is niacinamide, and a panthenol compound is panthenol.

8. The composition of any of the claim 1-3, wherein the first composition further comprises an aqueous carrier containing ethanol.

9. The composition of claim 8, wherein the first composition comprises from about 10% to about 90% of ethanol.

10. The composition of claim 8, wherein the first composition is substantially free of substantially insoluble oily compounds.

11. The composition of claim 8, wherein the first composition further comprising a thickener.

12. The composition of claim 11, wherein the thickener is Acrylates/C10-30 Alkyl Acrylate Crosspolymer.
